(19) United States
(12) Patent Application Publication
(10) Pub. No.: US 2008/0287986 A1
(43) Pub. Date: Nov. 20, 2008

(54) Closure device

(75) Inventors: Eric J. Thor, Arden Hills, MN (US); Michael J. Bonnette, Minneapolis, MN (US); Richard R. Prather, St. Michael, MN (US); Jason T. Anderson, Bloomington, MN (US)

Correspondence Address: GREGORY L BRADLEY;MEDRAD INC, ONE MEDRAD DRIVE, INDIANOLA, PA, 15051, US

(73) Assignee: Possis Medical, Inc.

(21) Appl. No.: 12/152,368

(22) Filed: May 14, 2008

Related U.S. Application Data
(60) Provisional application No. 60/930,829, filed on May 18, 2007.

(51) Int. Cl. A61B 17/08

(52) U.S. Cl. 606/213

(57) ABSTRACT

The present invention is for an arterial closure device which can be used to implement and augment the closure of a femoral artery or other related, adjacent or similar members of the vasculature and to reduce compression times associated therewith. A resorbable tubular plug is introduced through a delivery sheath subsequent to a procedure in which the delivery sheath is first utilized. The resorbable tubular plug is inserted through the delivery sheath and the distal tip of the resorbable tubular plug is positioned a short distance into the artery, whereby a suitable entry can be indicated by blood flow through and from the resorbable tubular plug. The delivery sheath is withdrawn to expose the resorbable tubular plug to the tissue track and to the arteriotomy and manual compression is applied to the wound site to foster and promote hemostasis.

### CROSS REFERENCES TO RELATED APPLICATIONS

This application claims priority from the earlier filed U.S. Provisional Application No. 60/930,829 filed May 18, 2007, entitled “Inserter”, and is hereby incorporated into this application by reference as if fully set forth herein.

### BACKGROUND OF THE INVENTION

1. Field of the Invention

The present invention is for a closure device which can be used to implement and augment closure of a femoral artery or other related, adjacent or similar members of the vasculature and to reduce compression times involved with such closure. Femoral arterial closure (or other closures) is required for every arterial intervention (diagnostic, cardiac, or peripheral). Despite the appearance of being a simple wrap-up to a complex procedure, such as by the use of a closure device or by the use of manual compression techniques, a femoral artery closure carries a serious procedural risk. Even with the introduction of various closure devices and with current manual compression techniques, bleeding remains a serious complication for interventional procedures. Closure devices are used in only twenty-five percent (25%) of the closure procedures, with manual compression used in the remainder where each method is used to repairingly seal the arteriotomy and the tissue track. The introduction of closure devices has given rise to new types of complications including embolization of closure components, suturing arteries closed, and permanent devices that prevent re-entry at a given site, etc. Although manual compression is the most frequently used closure technique, it is not popular, especially with the nursing staff. The bleeding complications are certainly one aspect of dissatisfaction in the use of either method. In addition, applying manual compression is uncomfortable and a lengthy process for both the patient and the surgical assistants.

The present invention pertains to disclosure describes a closure device for use in the closure of arteries including a resorbable tubular plug and an introducer sheath. The introducer sheath is the device through which all interventional or diagnostic equipment is introduced into the patient's arterial system. The introducer sheath is first passed along a tissue track, then into an arteriotomy, and then into an artery where one of many interventional vasculature procedures can be accomplished. Subsequently, the resorbable tubular plug is deployed along and through the in-place introducer sheath to enter a short distance into the artery. The introducer sheath is removed leaving the resorbable tubular plug in contact with the arteriotomy with the tissue track and within the artery. Compression of the site with the resorbable material in place is accomplished by manual pressure. By incorporating the delivery of the resorbable tubular plug through the introducer sheath, the closure process is greatly simplified and compression times are dramatically reduced. Finding the arteriotomy becomes a more automatic part of the procedure with the present device, as later described, as found by the use of a bleedback feature including a hole in the resorbable tubular plug. Furthermore, the ease in the use of the present device enables the utilization by the surgical staff and expedites the closure routine.

2. Description of the Prior Art

Artery closure, such as related to femoral artery closure, is required for all arterial interventional procedures including diagnostic procedures, coronary artery procedures, and peripheral arterial procedures. There is a variety of devices and techniques used to accomplish these arterial closures.

One internal device has a collagen plug and a resorbable foot, the latter of which is left in the artery post procedure. Collagen is drawn to the resorbable foot and hence to the arteriotomy via a suture. However, the resorbable foot sometimes embolized distally causing blockage of flow to the distal artery. Such an internal device used a bleedback port to indicate where the device is in relation to the artery. Nevertheless, positioning of such a the device can still be uncertain and doing so at the end of a procedure is not conducive to ease of use. Also, patient discomfort with large bulking agents pushed against the artery is a complaint common to one such device.

Another internal device was a balloon catheter which was positioned in the artery. A collagen matrix and collagen were injected into the tissue track once the artery was sealed with the balloon. Often, femoral clotting of the artery occurred when the balloon was improperly positioned and collagen and thrombin were injected into the artery. Also, with respect to positioning, the balloon was positioned in the artery, inflated and then pulled back until there was an evident resistance. This clearly has an uncertainty associated with the positioning of the balloon.

Yet another internal device included a nitinol device which pinched and closed the arteriotomy closed and which was permanent. For patients with peripheral artery disease, there may be a need for repeated interventions. The need to avoid any of these implantable nitinol devices for future interventions is undesirable.

Another internal device included the suturing and closure of the arteriotomy. Improper suturing where the suture extended to another wall of the artery has occurred using such a method.

For one or more of the aforementioned devices, and for other known and unmentioned devices, there are one or more difficulties to overcome. One such difficulty is that related to a bleeding complication rate where hemostasis is not achieved. Another difficulty is the risk of embolization of the closure component. Another difficulty is that of identifying the location of the arteriotomy.

Despite all of these optional devices, external manual compression remains the industry standard. Manual compression is often applied by a nurse/technician who applies finger tip compression on the wound site, once the introducer sheath is removed. Typical compression times require about 15 minutes to achieve hemostasis. In the case of an external compression (manual or device), the positioning problem is eliminated. However, applying a proper force becomes an issue. If too much force is applied, the femoral arterial blood flow can be disrupted (formation of clots, etc). If too little force is applied, bleeding will occur. Similarly, the femoral artery is in close proximity to the femoral vein, so the venous blood flow can be disrupted. An improperly positioned pressure can lead a hematoma where the blood pools internally since the pressure was not applied over the arteriotomy. In addition, a patient and nurse/technician discomfort is a significant negative effect against the use of this method. Nevertheless, the most significant issue with the use of this method is a failure to achieve hemostasis. With the rise of platelet inhibitors (Clopidigrel) and aspirin, the ability of the blood to form strong clots is degraded. Hence, the need to use something to augment manual compression is more important than ever. Clearly, a device which offers a significantly reduced compression time with easier-to-use components and procedures would be an advancement over the offerings of prior art devices.

### SUMMARY OF THE INVENTION

The general purpose of the present invention is to provide a closure device which can be used to implement and augment the closure of an artery such as a femoral artery or other related, adjacent or similar members of the vasculature and to reduce compression times.

According to one or more embodiments or illustrations of the present invention, there is provided an arterial closure device including a resorbable tubular plug and a delivery sheath, the latter of which can be used to deliver the resorbable tubular plug for deployment and use within the arteriotomy, the tissue track, and a short distance into an artery. The resorbable tubular plug is in the form of a tube which is open at the proximal end and closed at the rounded distal end. A hole which communicates with the lumen of the resorbable tubular plug is provided at a short distance proximal to the closed distal end of the resorbable tubular plug. The delivery sheath is in the form of a flexible tube, the proximal end of which secures to and extends distally from a configured connector fixture. The proximal end of the connector fixture is open to allow entry of the resorbable tubular plug into the delivery sheath. A flexible tube and valve are also connected to the connector fixture. Use of the present invention generally involves the insertion of the distal end of the delivery sheath through the tissue track and into the arteriotomy for use in the accomplishment of an invasive procedure involving insertion, use of, and withdrawal of interventional or diagnostic equipment, delivery of the resorbable tubular plug through the delivery sheath and into the artery, partial withdrawal of the resorbable tubular plug and full withdrawal of the delivery sheath to suitably position the resorbable tubular plug with respect to the arteriotomy, the tissue track and the artery, and manual application of pressure at the mutual site of the resorbable tubular plug, the arteriotomy, the artery, and the tissue track to achieve hemostasis.

One significant aspect and feature of the present invention is a closure device which can be used for implementing and augmenting closure of an artery such as a femoral artery or other related, adjacent or similar members of the vasculature.

Another significant aspect and feature of the present invention is a closure device used to significantly reduce compression times for artery closure.

Still another significant aspect and feature of the present invention is a closure device having a resorbable tubular plug and a delivery sheath.

Still another significant aspect and feature of the present invention is a closure device having a resorbable tubular plug for use with a delivery sheath.

Still another significant aspect and feature of the present invention is a closure device having a resorbable tubular plug which is delivered and placed within a tissue track, within an arteriotomy, and within and extending a short distance into an artery by the use and manipulation of a delivery sheath.

Still another significant aspect and feature of the present invention is a tubular resorbable tubular plug having a distal hole in communication with a lumen for sensing entry of the distal end of the resorbable tubular plug through the arteriotomy and into an artery as indicated by bleedback blood exiting the proximal end of the lumen.

Yet another significant aspect and feature of the present invention is the use of a distal hole in communication with a lumen for purging of air from the resorbable tubular plug to discourage or prevent a gas embolus.

Still another significant aspect and feature of the present invention is the use of fluoroscopy for sensing the entry of the distal end of the resorbable tubular plug through the arteriotomy and into an artery.

Still another significant aspect and feature of the present invention is redundancy as provided by observed bleedback blood or by the use of fluoroscopy to determine the positions of the delivery sheath and resorbable tubular plug.

Still another significant aspect and feature of the present invention is the use of a resorbable tubular plug of cellulose with or without starch, of collagen or other quick acting resorbable material to promote and foster hemostasis.

Still another significant aspect and feature of the present invention is a resorbable tubular plug which can be constructed of PVA and sugar in various ratios of combination, including resorbable tubular plugs which can be constructed without the use of a sugar, either of which can have different wall thicknesses and dissolving rates, which can be provided to meet the needs of a particular surgical application to promote and foster hemostasis.

Still another significant aspect and feature of the present invention is the use of a cellulose top coating, which is used as a temporary hydrophilic coating, residing on the delivery sheath to aid insertion.

Still another significant aspect and feature of the present invention is a resorbable tubular plug which can be used with other types, lengths and sizes of introducer sheaths.

Having thus described embodiments of the present invention and having set forth significant aspects and features of the present invention, it is the principal object of the present invention to provide an arterial closure device.

### BRIEF DESCRIPTION OF THE DRAWINGS

Other objects of the present invention and many of the attendant advantages of the present invention will be readily appreciated as the same becomes better understood by reference to the following detailed description when considered in connection with the accompanying drawings, in which like reference numerals designate like parts throughout the figures thereof and wherein:

FIG. 1 is an exploded isometric view of the arterial closure device, the present invention;

FIG. 2 is an assembled isometric view of the arterial closure device of FIG. 1;

FIG. 3 is an exploded view in cross section of a delivery sheath, a connector fixture, a resorbable tubular plug, one end of a flexible tube, and a full side view of a valve;

FIG. 4 is an assembled view in cross section of the delivery sheath, the connector fixture, the resorbable tubular plug, one end of the flexible tube, and a full side view of the valve of FIG. 3;

FIG. 5 is a side view of the delivery sheath having been inserted through and residing in the tissue track, and thence into and through an arteriotomy to extend into and along a short distance along an artery;

FIG. 6 illustrates the post-medical procedure phase of the mode of operation of the present invention, whereby the resorbable tubular plug has been delivered through the delivery sheath;

FIG. 7 illustrates the delivery sheath having been manually and proximally repositioned and withdrawn fully from the artery, the arteriotomy, and the tissue track;

FIG. 8 illustrates complete removal of the delivery sheath from engagement with the resorbable tubular plug and the application of pressure at the wound site by one or more fingers;

FIG. 9 illustrates the distal region of the resorbable tubular plug as influenced by applied pressure by one or more fingers at the wound site and as influenced by contact with blood or other fluids to achieve hemostasis within the tissue track and within the arteriotomy; and,

FIG. 10 illustrates resulting hemostasis along and within the tissue track and at the arteriotomy.

### DETAILED DESCRIPTION OF THE PREFERRED EMBODIMENTS

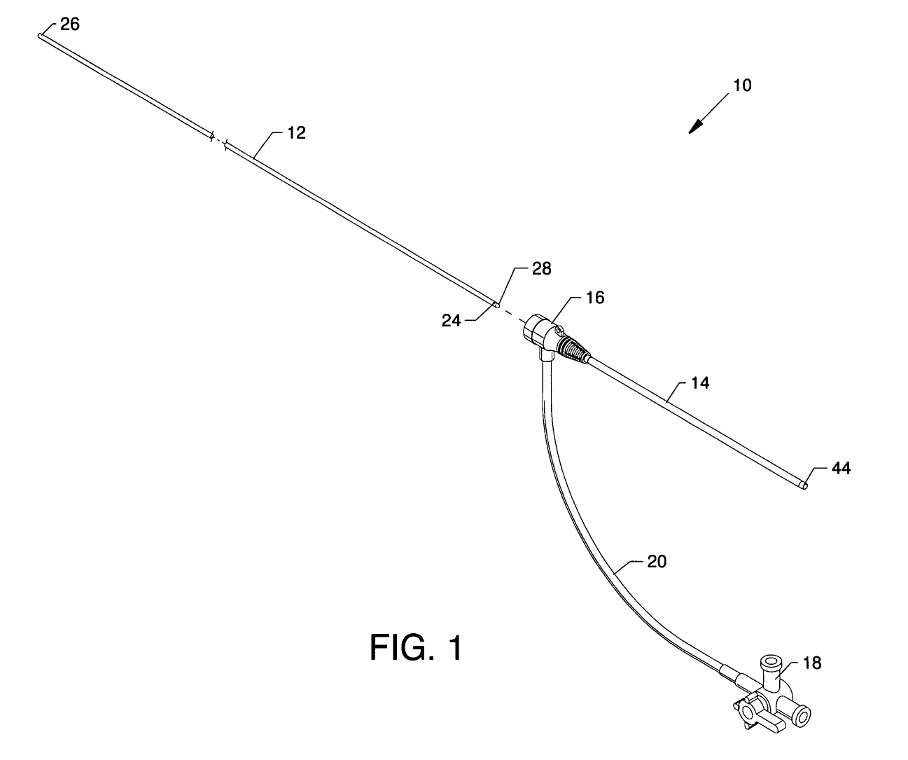

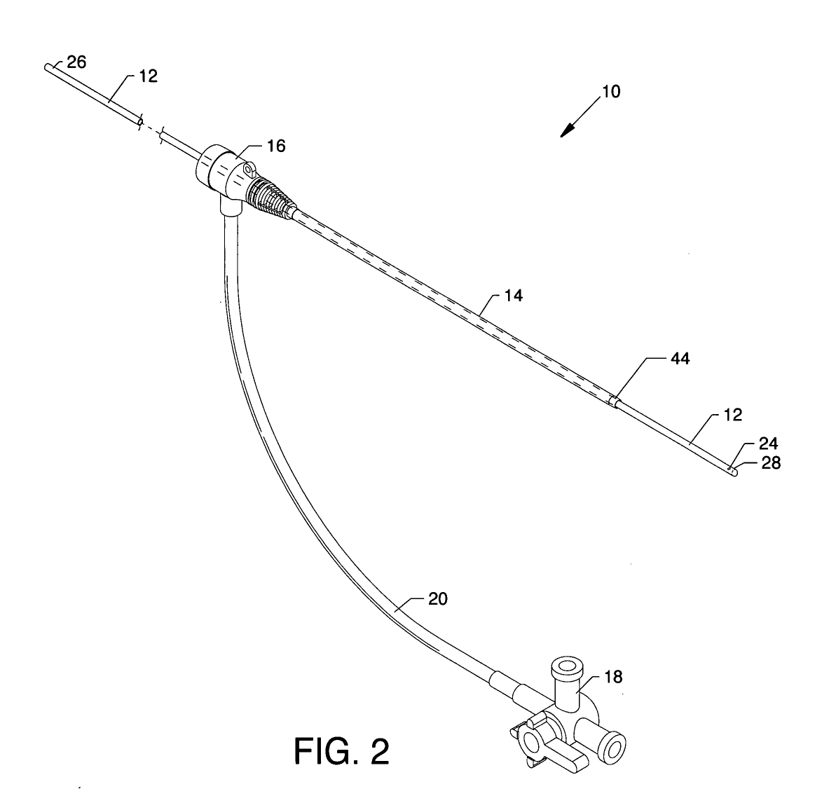

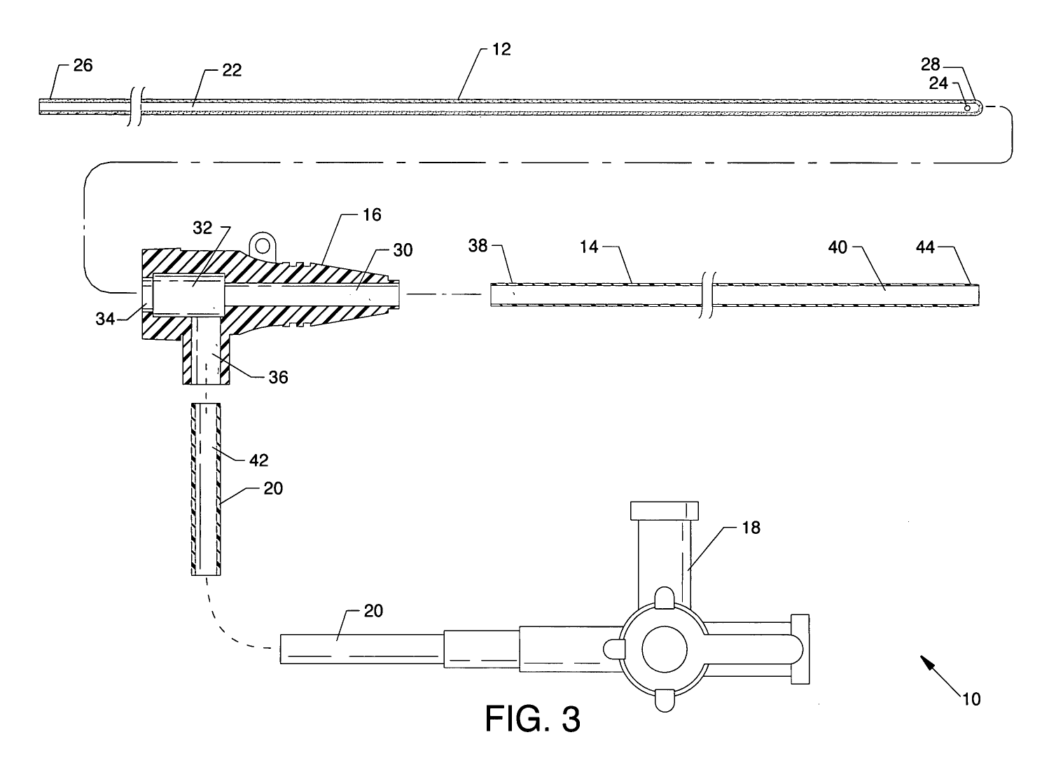

FIG. 1 is an exploded isometric view and FIG. 2 is an assembled isometric view of the arterial closure device 10, the present invention, which includes a resorbable tubular plug 12, a delivery sheath 14 in the form of a flexible tube, a connector fixture 16, and a valve 18 connected to the connector fixture 16 by a flexible tube 20. The resorbable tubular plug 12 can be made of cellulose with or without starch, collagen, a combination of sugar and polyvinyl alcohol (PVA) or other quickly resorbable materials. The use of cellulose or other suitable material provides a hemostatic agent to aid in rapid hemostasis. The resorbable tubular plug 12 is in the form of a tube and includes a lumen 22 (FIG. 3), and also includes a hole 24 extending through the wall of the resorbable tubular plug 12 providing communication between the lumen 22 and the environment exterior to the resorbable tubular plug 12. The resorbable tubular plug 12 includes an open proximal end 26 and a closed distal end 28 located at the respective ends of the lumen 22. The hole 24 is located a short distance proximal to and in close proximity to the closed distal end 28 of the resorbable tubular plug 12. The distal end 28 is rounded to facilitate entry into the delivery sheath 14 and for ease of passage through the delivery sheath 14.

A preferred composition for the resorbable tubular plug 12 is 1 gram of PVA (provided in sheet form) and 1 gram of sugar (sucrose) dissolved in 10 grams of water. That is, one part by weight of PVA to one part by weight of sucrose. An alternative preferred composition is 1 gram of PVA (provided in sheet form) and 2 grams of sugar (sucrose) dissolved in 10 grams of water. That is, one part by weight of PVA to two parts by weight of sucrose. From these dissolved compositions, suitable and preferred resorbable tubular plugs 12 may be prepared. One method of preparation is by repeatedly dipping of a silicone tube having a 0.070 inch OD in one of the PVA and sucrose compositions dissolved in water. The dissolved composition coats the tube and then the water is allowed to evaporate, thereby producing a resorbable tubular plug with an inner diameter (ID) of about 0.070 inch. Resorbable tubular plugs can be made in this manner to provide inner diameters of about 0.005 inch to about 0.080 inch by providing silicone tubes of like outer diameter as forms for dipping. Wall thickness of from about 0.005 inch to about 0.040 inch can be prepared, if necessary, using repeated dippings—more preferably, wall thickness may be from 0.007 inch to about 0.013 inch. Dissolution of the resorbable tubular plugs into water can serve as a simple model system to test for approximate resorbing time. The useable time for the tubes for either composition can be increased by increasing wall thickness and useable time decreases with increasing sugar proportions. For example, resorbable tubular plugs of 1:1 or 1:2 (PVA:sucrose) with wall thickness of about 0.01 inch dissolve in roughly 4 minutes. Resorbable tubular plugs of 1:2 composition with a wall thickness 0.03 inch dissolve in roughly 13 minutes, while resorbable tubular plugs of 1:1 composition dissolve in roughly 17 minutes. For comparison purposes, PVA tubes (no sugar added) of roughly 0.01 inch wall thickness dissolve in roughly 10 minutes, while PVA tubes (no sugar added) of roughly 0.02 inch wall thickness dissolve in roughly 20 minutes. Thus, it can be seen that control of dissolution (which simulates the rate of resorbing) of the resorbable tubular plugs can be adjusted to the needs of the particular surgical application by adjusting the composition and thickness of the walls for a given inner diameter. Moreover, by varying wall thickness, portions of the resorbable tubular plug can be made stronger or weaker, as desired, and can resorb more quickly or more slowly, as desired. In one particular exemplary prototype resorbable tubular plug configuration, a PVA (no sugar added) resorbable tubular plug was prepared with a 0.070 inch inner diameter and a distal end wall thickness of about 0.008 inch and a proximal wall thickness of about 0.013 inch was successfully inserted into a test animal. Optionally, a cellulose coating can be added to the resorbable tubular plug to provide a temporary hydrophilic coating to both delay resorption and/or to keep the outside surface of the resorbable tubular plug 12 from becoming sticky and thereby harder to manipulate through the delivery sheath. It will be understood that alternative methods of plug production might be used, particularly when desirable compositions and thickness are standardized and larger more economical quantities are required. For example, extrusion or pultrusion or other large scale production methods might be effectively adopted. Control of the configuration, in terms of composition, inner diameter, wall thickness and profile over the lineal extent of the resorbable tubular plugs 12 allows for selection of sufficient initial stiffness or sufficient spine for a sufficient initial time period, such that the resorbable tubular plug 12 may be entered into and passed partially through the lumen of the delivery sheath 14 to an intended location extending from the exterior of the patient, through the tissue path, through the arteriotomy site and into the artery and then allow withdrawal of the delivery sheath 14. In the alternative, the resorbable tubular plug 12 might also include antibiotics and/or drugs within the composition of efficacious amounts.

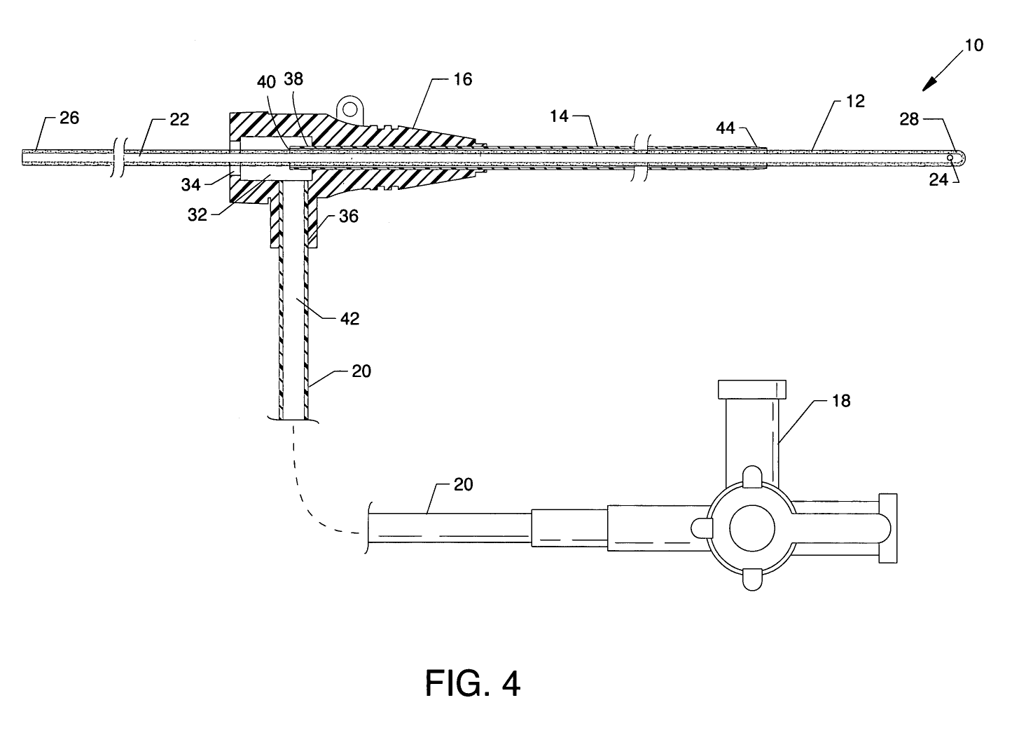

FIG. 3 is an exploded view in cross section, and FIG. 4 is an assembled view in cross section of the delivery sheath 14, the connector fixture 16, the resorbable tubular plug 12, one end of the flexible tube 20 and a full side view of the valve 18. The connector fixture 16 includes a distally located longitudinally oriented bore 30 aligned with and connected to a centrally located longitudinally oriented cavity 32. A proximally located opening 34 is aligned with, connected to, and communicates with the centrally located cavity 32. Another bore 36 is aligned perpendicularly to, connected to, and communicates with the cavity 32. A proximal end 38 of the delivery sheath 14 is aligned within and suitably secured within the bore 30, whereby a lumen 40 of the delivery sheath 14 communicates with the cavity 32, as well as with a lumen 42 of the flexible tube 20. A cellulose top coat (not shown) can be applied over the sheath 14 to function as a temporary hydrophilic coating in order to aid the insertion of the sheath 14 into the arteriotomy and the tissue track. The cellulose top coat of the sheath 14 is dissolved into the vasculature prior to the end of the procedure using the present invention. The resorbable tubular plug 12 can extend through and align with the opening 34 and the cavity 32 of the connector fixture 16. The resorbable tubular plug 12 can also align within the lumen 40 of the delivery sheath 14 and extend beyond the distal end 44 of the delivery sheath 14.

### Mode of Operation

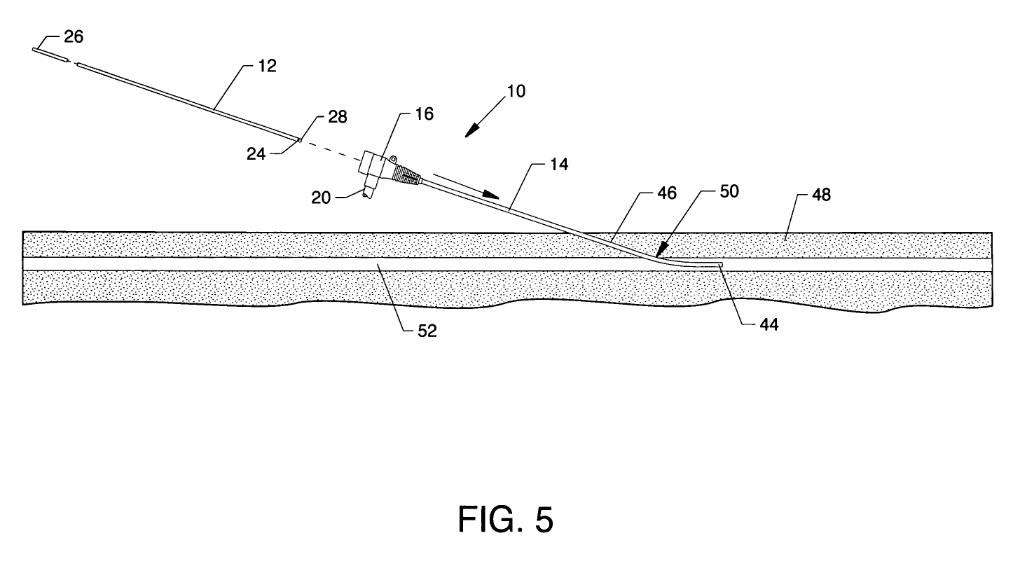

FIGS. 5-10 illustrate the mode of operation of the closure device 10 shown in use with a tissue track 46 extending through tissue 48 to enter an arteriotomy 50 extending through the wall of an artery 52.

In FIG. 5, the delivery sheath 14 is shown having been inserted through and residing in the tissue track 46 and thence into and through the arteriotomy 50 to extend a short distance along the artery 52 for subsequent use in accomplishing one or more medical procedures, whereby interventional or diagnostic equipment can be introduced into the patients arterial system. Positioning of the distal end 44 of the delivery sheath 14 can be monitored by the use of fluoroscopy or other methods known to the art. The resorbable tubular plug 12 is shown being available for entry into and passage through the delivery sheath 14 via the connector fixture 16 after medical procedures have been accomplished.

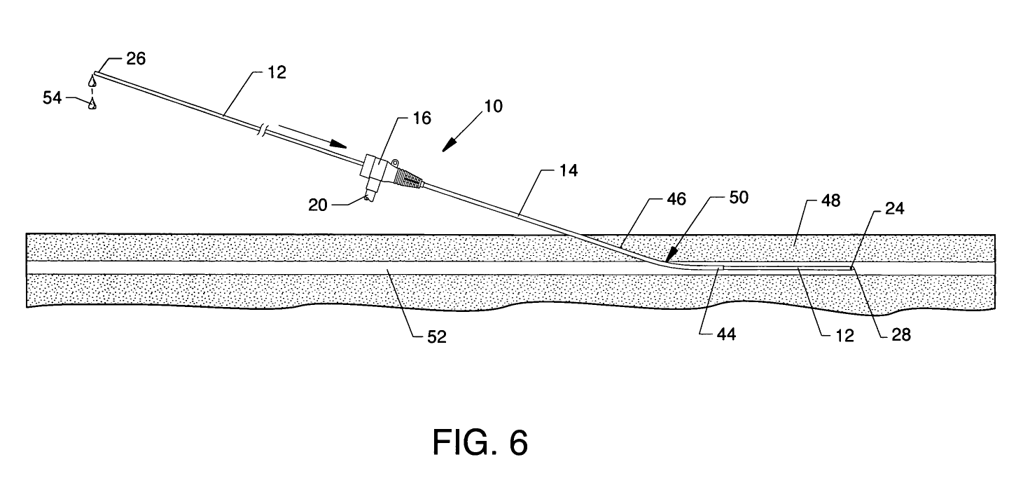

FIG. 6 illustrates the post-medical procedure phase of the mode of operation of the present invention where the resorbable tubular plug 12 has been delivered through the delivery sheath 14 to extend a short distance within and along the artery 52.

Subsequent to the withdrawal of the interventional or diagnostic equipment from the artery 52, the delivery sheath 14, and connector fixture 16, the rounded closed distal end 28 of the resorbable tubular plug 12, and thus the resorbable tubular plug 12, is introduced and advanced distally to pass directly through the opening 34 and through the cavity 32 of the connector fixture 16 and into the lumen 40 of the delivery sheath 14. The resorbable tubular plug 12 is separated from direct contact with the tissue track 46 and the arteriotomy 50 by the delivery sheath 14, whereby the resorbable tubular plug 12 passes indirectly but in close proximity through the locale of the tissue track 46 and the arteriotomy 50 and is advanced distally to extend a short distance beyond the distal end 44 of the delivery sheath 14 and directly into the artery 52. The resorbable tubular plug 12 is advanced distally until the hole 24 near the distal end 28 of the resorbable tubular plug 12 is positioned a short distance beyond the distal end 44 of the delivery sheath 14 into the artery 52, wherein bleedback blood 54 exiting the proximal end 26 of the resorbable tubular plug 12 indicates suitable positioning of the resorbable tubular plug 12 in the artery 52. Blood in the artery 52 is sent through the hole 24 by vascular system pressure and along the lumen 22 to exit as bleedback blood 54 at the proximal end 26 of the resorbable tubular plug 12. Thus, a central portion of the resorbable tubular plug 12 is positioned along the interior of the connector fixture 16 and the delivery sheath 14 and a distal portion of the resorbable tubular plug 12 is positioned along and within a short portion of the artery 52. The proximal portion of the resorbable tubular plug 12 is shown extending proximal to the connector fixture 16.

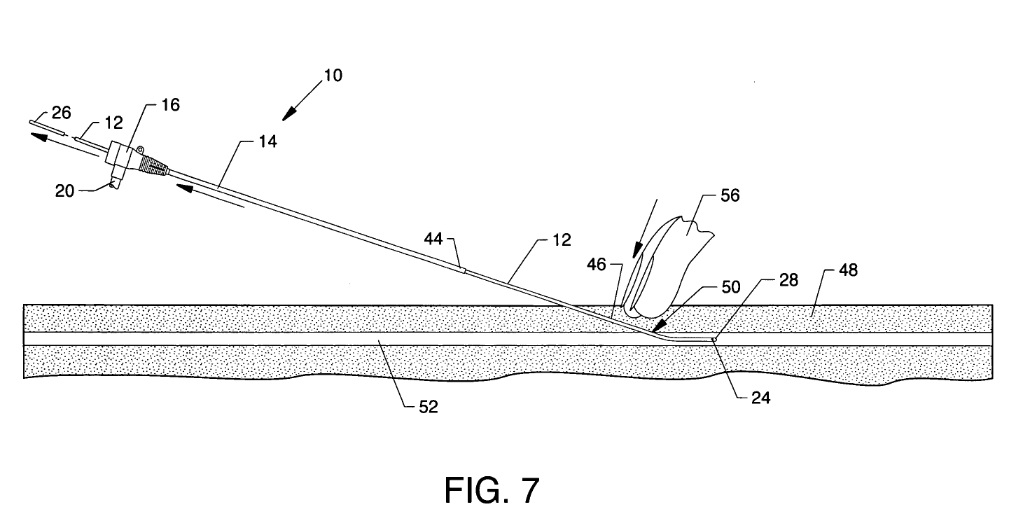

FIG. 7 illustrates the delivery sheath 14 having been manually and proximally repositioned and withdrawn fully from the artery 52, the arteriotomy 50, and the tissue track 46, and also illustrates the resorbable tubular plug 12 having been manually and proximally repositioned to suitably locate the resorbable tubular plug 12 within the arteriotomy 50 and the tissue track 46. Repositioned withdrawal in a proximal direction of the delivery sheath 14 is effected until the distal end 44 of the delivery sheath 14 discontinues contact with the tissue track 46. Repositioning of the delivery sheath 14 also discontinues coverage of the resorbable tubular plug 12 within the tissue track 46, whereby a portion of the resorbable tubular plug 12 is exposed to the surrounding tissue track 46 to interact therewith, as described later in detail. Preferably, such repositionings can be accomplished simultaneously by supportively using nominal manual pressure applied externally by one or more fingers 56 to the wound site including the tissue 48, the resorbable tubular plug 12, the arteriotomy 50, the artery 52, and the tissue track 46. In the alternative, individual repositionings can be utilized instead of simultaneous repositioning.

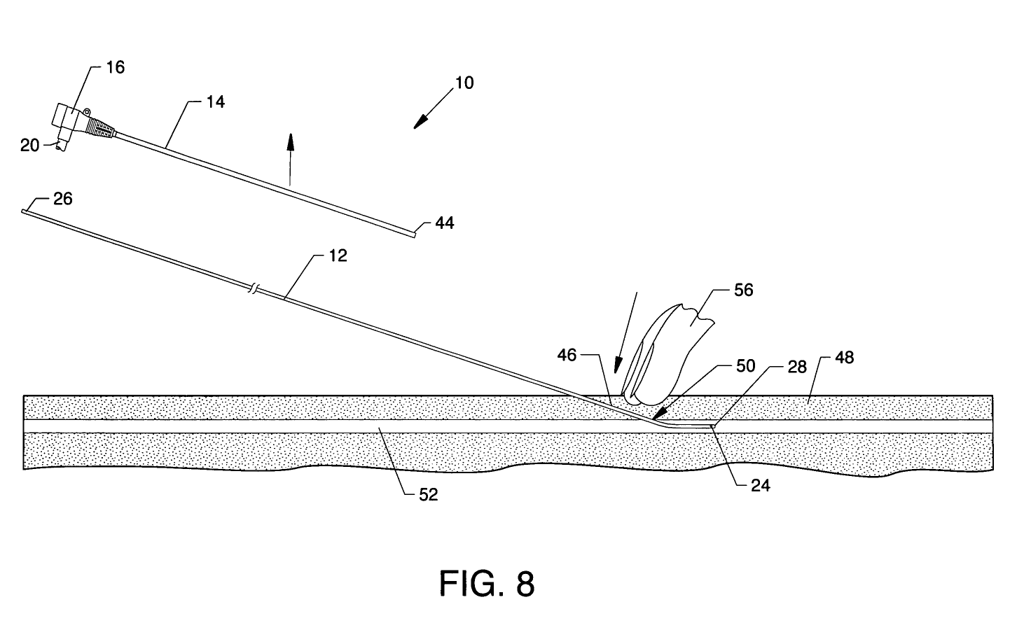

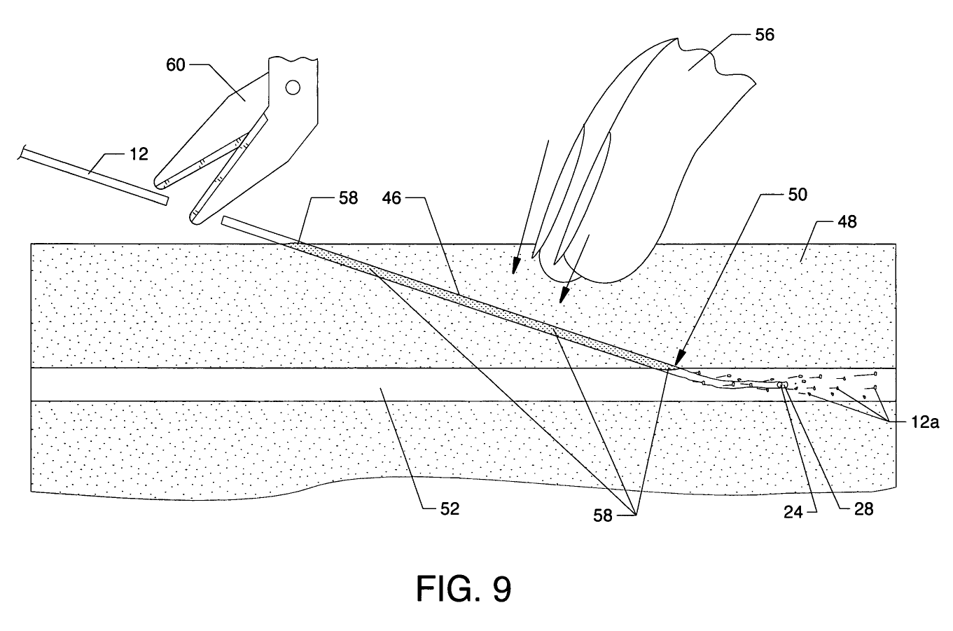

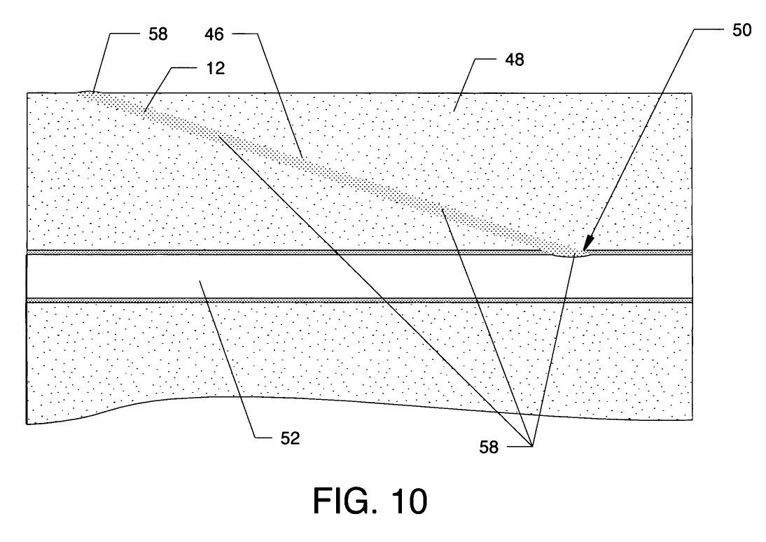

FIG. 8 illustrates the complete removal of the delivery sheath 14 from engagement with the resorbable tubular plug 12. Pressure by one or more fingers 56 is maintained at the wound site until hemostasis is achieved as described with reference to FIGS. 9 and 10.

FIG. 9 illustrates the distal region of the resorbable tubular plug 12 as influenced by applied pressure by one or more fingers 56 at the wound site and as influenced by contact with blood or other fluids to achieve hemostasis 58 within the tissue track 46 and within the arteriotomy 50. Typically, pressure is applied for 2-6 minutes to allow hemostasis to occur. The distal end 28 of the resorbable tubular plug 12 and a short portion of the resorbable tubular plug 12 extending into the artery 52 is subjected to moistening by blood or other fluids resulting in erosion, breakdown, dissolving, loosening and carrying away of multiple resorbable plug particles 12a or dissolvent along the artery 52 by blood flow within the artery 52. Reaction of the material comprising the resorbable tubular plug 12 with blood or others fluids aids, promotes and speeds the hemostasis formation in the arteriotomy 50. The distal portion of the resorbable tubular plug 12, which is in direct contact with the tissue track 46, is moistened sufficiently by contact with blood or other fluids residing in the tissue 48 to cause breakdown, dissolving, softening and reshaping of the resorbable tubular plug 12 in reaction with the material comprising the resorbable tubular plug 12 in order to foster, promote and speed hemostasis 58 within the tissue track 46, and can also contribute to and foster hemostasis 58 in the adjacent arteriotomy 50. During moisturizing of the resorbable tubular plug 12, lumen 22 of the resorbable tubular plug 12, which has already served the purpose of transporting bleedback blood 54 proximally therealong, is deformed and reshaped due to the applied manual pressure and the moisturizing thereof and may no longer at this stage function as a lumen. The remaining external portion of the resorbable tubular plug 12 can be trimmed in close proximity to the exterior of the tissue 48, such as by the use of a surgical scissors 60 or another suitable instrument.

FIG. 10 illustrates the resulting hemostasis 58 along and within the tissue track 46 and at the arteriotomy 50 where the resorbable tubular plug 12 has dissolved to foster, promote and to form the hemostasis 58.

One major advantage of the use of the present invention is that it is very easy to use. Combining the delivery sheath 14 with the resorbable tubular plug 12 as a packaged unit is a matter of convenience. In the alternative, other delivery sheaths known in the art can be utilized with an individually packed resorbable tubular plug 12 of the present invention. In most procedures involving insertion, use of, and withdrawal of interventional or diagnostic equipment, the physician has to position an introducer anyway. The nursing staff can administer the present invention versus other types of closure devices which often require a physician to administer. Also, the present invention is using an industry standard method of closure by providing manual compression. By leaving resorbable material in the tissue track 46, the efficacy and speed of manual compression closure is dramatically improved. Moreover, the resorbable material acts as a glue or bonding material relative to the adjacent tissue along the tissue path. Further, the resorbable material also acts as a gluing or bonding material at the arteriotomy site. The blood within the tissue track sealingly interacts with the composition material.

The use of a relatively quick resorbing material greatly reduces the risk of embolization. Even if a loosened distal portion of the resorbable tubular plug 12 ends up in the distal artery 52, the resorbable material will most likely be resorbed by the end of the procedure. For the resorbing material in the tissue track 46, the resorption is slow enough that it provides a benefit to manual compression. The resorption in the tissue track 46 is slow since it is not exposed to a swift blood flow as in the artery 52, and furthermore, the protection by the delivery sheath 14 inhibits resorption until such a time when the delivery sheath 14 is removed from the tissue track 46.

Furthermore, use of the present invention should cause less pain for the patient. One prior art device leaves a bulking agent at the arteriotomy resulting in pain. While manual compression is certainly an uncomfortable experience, the resorbable tubular plug 12 is small and pliable enough that it will not be a painful lump in the patient. Furthermore, compression times are reduced so that pain exposure time is dramatically reduced.

Use of the present invention is safe. Positioning error is not that critical since the resorbable material resorbs quickly. The resorbable material seems efficacious, but if for some reason the resorbable material is not properly introduced into the tissue track 46, manual compression will be used anyway, but will require a longer time for application of compression. Additionally, the boosted sealing power of the resorbable material should reduce bleeding complications from that of manual compression alone.

Various modifications can be made to the present invention without departing from the apparent scope thereof.

### PARTS LIST

10 arterial closure device
12 resorbable tubular plug
12a resorbable plug particles
14 delivery sheath
16 connector fixture
18 valve
20 flexible tube
22 lumen
24 hole
26 proximal end
28 distal end
30 bore
32 cavity
34 opening
36 bore
38 proximal end
40 lumen
42 lumen
44 distal end
46 tissue track
48 tissue
50 arteriotomy
52 artery
54 bleedback blood
56 finger
58 hemostasis
60 surgical scissors

1. An arterial closure device comprising:
a. a connector fixture with a central bore therethrough, said connector fixture having a distal end and a proximal end;
b. an elongated tubular delivery sheath having a proximal end and a distal end, said proximal end of said elongated tubular delivery sheath extending within said central bore of said connector fixture and affixed therewithin, said distal end of said elongated tubular sheath extending beyond said distal end of said connector fixture; and,
c. an elongated resorbable tubular plug having a proximal end and a distal end, said elongated resorbable tubular plug extending through said bore of said connector fixture and through said elongated tubular delivery sheath, said elongated resorbable tubular plug being closed at its distal end and open at its proximal end and said elongated resorbable tubular plug having a hole near and proximal to said closed distal end.

2. The arterial closure device of claim 1, wherein said elongated tubular delivery sheath and said elongated resorbable tubular plug are flexible.

3. The arterial closure device of claim 2, wherein said elongated resorbable tubular plug is slideable within and through said elongated tubular sheath and said bore of said connector fixture.

4. The arterial closure device of claim 3, wherein said elongated resorbable tubular plug is made from a material selected from the group consisting of cellulose with starch, cellulose without starch, a collagen, a combination of sugar and PVA, and other quickly resorbable materials.

5. The arterial closure device of claim 4, wherein said distal end of said elongated resorbable tubular plug is extendable beyond said distal end of said elongated tubular delivery sheath.

6. The arterial closure device of claim 5, wherein said elongated tubular delivery sheath has a cellulose coating on an along its outer surface.

7. The arterial closure device of claim 6, wherein said central bore of said connector fixture is coaxial with a cavity and an opening proximal to said central bore.

8. The arterial closure device of claim 7, wherein said connector fixture has an extension perpendicular to said cavity, said perpendicular extension having a central bore therein, an elongated flexible tube having a proximal end and a distal end, said proximal end of said elongated flexible tube extending within said central bore of said extension and affixed therein, and said distal end of said elongated flexible tube being connected to a valve.

9. A method for closing an incision to an artery comprising the steps of:
a. inserting an elongated resorbable tubular plug with a closed distal end and an open proximal end into said incision through a tissue track and into a specific locality within said artery;
b. withdrawing bleedback blood from said artery from a hole near said distal end of said elongated resorbable tubular plug outwardly from said open proximal end of said elongated resorbable tubular plug which indicates that said distal end of said elongated resorbable tubular plug is within said artery;
c. applying manual pressure externally for a short time to said tissue track through which said elongated resorbable tubular plug passes between said incision and said artery; and,
d. cutting off said elongated resorbable tubular plug external to said incision.

10. The method of claim 9, wherein said elongated resorbable tubular plug is made from a material selected from the group consisting of cellulose with starch, cellulose without starch, a collagen, a combination of sugar and PVA, and other quickly resorbable materials.

11. The method of claim 9, wherein surgical scissors are used for cutting off said elongated resorbable tubular plug external to said incision.

12. The method of claim 9, wherein an elongated tubular delivery sheath, having a proximal end and a distal end, is initially inserted into said incision through tissue surrounding said artery and into said artery and thence said elongated resorbable tubular plug is slidingly inserted into said elongated tubular delivery sheath and extended from said distal end of said elongated tubular delivery sheath into said artery.

13. The method of claim 12, wherein, subsequent to the step of withdrawing bleedback blood from said artery, said elongated tubular delivery sheath is withdrawn externally from said incision.

14. A method for facilitating hemostasis of an incision to an artery comprising the steps of:
a. inserting an elongated resorbable tubular plug with a closed distal end and an open proximal end into said incision through a tissue track and into a specific locality within said artery;
b. withdrawing bleedback blood from said artery from a hole near said distal end of said elongated resorbable tubular plug outwardly from said open proximal end of said elongated resorbable tubular plug which indicates that said distal end of said elongated resorbable tubular plug is within said artery;
c. applying manual pressure externally for a short time to said tissue track through which said elongated resorbable tubular plug passes between said incision and said artery whereby hemostasis within said tissue track occurs; and,
d. cutting off said elongated resorbable tubular plug external to said incision.

15. The method of claim 14, wherein said elongated resorbable tubular plug is made from a material selected from the group consisting of cellulose with starch, cellulose without starch, a collagen, a combination of sugar and PVA, and other quickly resorbable materials.

16. The method of claim 14, wherein surgical scissors are used for cutting off said elongated resorbable tubular plug external to said incision.

17. The method of claim 14, wherein an elongated tubular delivery sheath, having a proximal end and a distal end, is initially inserted into said incision through tissue track surrounding said artery and into said artery and thence said elongated resorbable tubular plug is slidingly inserted into said elongated tubular delivery sheath and extended from said distal end of said elongated tubular delivery sheath into said artery.

18. The method of claim 17, wherein, subsequent to the step of withdrawing bleedback blood from said artery, said elongated tubular delivery sheath is withdrawn externally from said incision.

19. The arterial closure device of claim 4, wherein said elongated resorbable tubular plug is made from a composition of PVA and sugar (sucrose), 1:1 or 1:2 (PVA:sucrose).

20. The method of claim 10, wherein said elongated resorbable tubular plug is made from a composition of PVA and sugar (sucrose), 1:1 or 1:2 (PVA:sucrose).

21. The method of claim 15, wherein said elongated resorbable tubular plug is made from a composition of PVA and sugar (sucrose), 1:1 or 1:2 (PVA:sucrose).

22. An arterial closure device combination for an arteriotomy, the device combination comprising:
a. a resorbable tubular plug; and,
b. a delivery sheath, the delivery sheath having a lumen capable of allowing passage of the resorbable tubular plug therethrough.

23. The device combination of claim 22, wherein the resorbable tubular plug is tubular and includes an open proximal end and a closed distal end.

24. The device combination of claim 23, wherein the closed distal end of the resorbable tubular plug is rounded.

25. The device combination of claim 23, wherein the resorbable tubular plug further includes a communication hole, the communication hole situated a short distance proximal to the closed distal end.

26. The device combination of claim 22, wherein the lumen of the delivery sheath is in a flexible tube, the tube having a proximal end secured to and extending distally from a configured connection fixture.

27. The device combination of claim 26, wherein the configured connection fixture includes a proximal end and wherein the proximal end of the connection fixture is open to allow entry and passage of the resorbable tubular plug through the lumen of the delivery sheath.

28. The device combination of claim 27, wherein the connection fixture includes a valve.

29. The device combination of claim 28, wherein the valve of the connection fixture includes another flexible tube connected thereto.

30. The device combination of claim 23, wherein the resorbable tubular plug includes PVA (polyvinyl alcohol).

31. The device combination of claim 23, wherein the resorbable tubular plug includes at least one compound selected from the group consisting of PVA (polyvinyl alcohol), cellulose, starch, sucrose, and collagen, and further wherein the resorbable tubular plug optionally includes salt, an antibiotic or a drug.

32. A method of surgery comprising the steps of:
a. providing a delivery sheath, the delivery sheath having a lumen with a distal end situated within an artery and a proximal end situated external to a patient, the lumen passing through a tissue path and an arteriotomy site;
b. providing a resorbable tubular plug and passing the resorbable tubular plug partially through the lumen such that the resorbable tubular plug extends through the arteriotomy site and into the artery;
c. withdrawing the delivery sheath while leaving the resorbable tubular plug extending through the arteriotomy site and into the artery; and,
d. applying pressure to the arteriotomy site for a period of time sufficient to stop bleeding through the arteriotomy site with the resorbable tubular plug extending therethrough.

33. The method of claim 32, wherein the resorbable tubular plug, extending through the arteriotomy site and into the artery, also extends into the tissue path.

34. The method of claim 33, wherein the resorbable tubular plug, extending through the arteriotomy site and into the artery and into the tissue path, also extends outward from the tissue path.

35. The method of claim 34, wherein the resorbable tubular plug is tubular and includes an open proximal end and a closed distal end.

36. The method of claim 35, wherein the closed distal end is rounded.

37. The method of claim 34, wherein the resorbable tubular plug further includes a communication hole, the communication hole situated a short distance proximal to the closed distal end, the communication hole allowing fluid communication between the artery and the proximal end of the resorbable tubular plug.

38. The method of claim 37, and further comprising the step of:
a. detecting position of the communication hole in the artery by observing blood exiting the proximal end of the resorbable tubular plug.

39. The method of claim 38, wherein the blood exiting the proximal end of the resorbable tubular plug is driven by arterial vascular pressure from within the artery, thereby purging the resorbable tubular plug of air.

40. The method of claim 32, wherein the lumen of the delivery sheath is in a flexible tube, the tube having proximal end external to the patient secured to and extending distally from a configured connection fixture.

41. The method of claim 40, wherein the configured connection fixture includes a proximal end and wherein the proximal end of the connection fixture is open to allow entry and passage of the resorbable tubular plug through the lumen of the delivery sheath.

42. The method of claim 41, wherein the connection fixture includes a valve.

43. The method of claim 42, wherein the valve of the connection fixture includes another flexible tube connected thereto.

44. The method of claim 42, wherein the delivery sheath has been previously used to pass diagnostic or interventional devices into the patient.

45. The method of claim 32, wherein the resorbable tubular plug includes PVA (polyvinyl alcohol).

46. The method of claim 32, wherein the resorbable tubular plug includes at least one compound selected from the group consisting of PVA (polyvinyl alcohol), cellulose, starch, sucrose, and collagen.

47. The method of claim 32, wherein the resorbable tubular plug is configured to include sufficient spine for a sufficient initial time period, such that it may be entered into and passed partially through the lumen of the delivery sheath to an intended location extending from the exterior of the patient, through the tissue path, through the arteriotomy site and into the artery and then allow withdrawal of the delivery sheath.

48. The method of claim 47, wherein the resorbable tubular plug is tubular and the configuration of the resorbable tubular plug includes the inner diameter of the tubular plug, the tubular plug composition and the wall thickness.

49. The method of claim 48, wherein the tubular resorbable tubular plug has an inner diameter of about 0.005 inch to about 0.080 inch.

50. The method of claim 49, wherein the tubular resorbable tubular plug has a wall thickness of from about 0.005 inch to about 0.040 inch.

51. The method of claim 50, wherein the wall thickness is from about 0.007 inch to about 0.013 inch and the resorbable tubular plug has an inner diameter of about 0.005 inch to about 0.080 inch.

52. The method of claim 48, wherein the wall thickness is varied according to the spine required along the length of the resorbable tubular plug.

53. The method of claim 52, wherein the distal end of the resorbable tubular plug has a wall thickness of about 0.008 inch and the proximal end of the resorbable tubular plug has a wall thickness of about 0.013 inch and the resorbable tubular plug has an inner diameter of about 0.005 inch to about 0.080 inch and the delivery sheath is a 7 French size.

54. The method of claim 53, wherein the composition of the resorbable tubular plug is from a composition of about 1 part by weight PVA to 1 part by weight sucrose to a composition of about 1 part by weight PVA to about 2 parts by weight sucrose.

55. The method of claim 32, wherein the artery is a femoral artery.

56. The method of claim 32, wherein homeostasis results from the pressure application.

57. The method of claim 32, wherein pressure is applied of about 2-6 minutes.

58. The method of claim 32, wherein hemostasis results from the material of the resorbable tubular plug acting as a gluing material to tissue adjacent the tissue path.

59. The method of claim 58, wherein the resorbable material of the resorbable tubular plug sealingly interacts with the blood in the tissue track.

60. A resorbable tubular plug having a tubular shape with a closed distal end and an open proximal end and a communication hole just proximal of the distal end.

61. The resorbable tubular plug of claim 60, wherein the resorbable tubular plug has sufficient spine for a sufficient initial time period, such that it may be entered into and passed partially through the lumen of a delivery sheath to an intended location extending from exterior of the patient, through a tissue path, through an arteriotomy site and into an artery and then allow withdrawal of the delivery sheath, leaving the resorbable tubular plug in the intended location.

62. The resorbable tubular plug of claim 61, wherein pressure applied for 2-6 minutes allow hemostasis to occur.
